(19) United States
(12) Patent Application Publication
(10) Pub. No.: US 2004/0158078 A1
(43) Pub. Date: Aug. 12, 2004

(54) Method for producing pyrrolidones

(76) Inventors: Rolf-Hartmuth Fischer, Heidelberg (DE); Rolf Pinkos, Bad Durkheim (DE); Markus Rosch, Oppenheim (DE); Frank Stein, Bad Durkheim (DE)

Correspondence Address: KEIL & WEINKAUF, 1350 CONNECTICUT AVENUE, N.W., WASHINGTON, DC, 20036, US

(21) Appl. No.: 10/481,470

(22) PCT Filed: Jun. 18, 2002
(86) PCT No.: PCT/EP02/06722

(30) Foreign Application Priority Data
Jun. 19, 2001 (DE) 101 29 336.4

(51) Int. Cl. C07D 207/267

(52) U.S. Cl. 548/554

(57) ABSTRACT

In a process for preparing pyrrolidone, which may be N-substituted, substrates selected from among C₄-dicarboxylic acids and their derivatives are hydrogenated in the gas phase under anhydrous conditions with or without addition of ammonia or primary amines and using a Cr-free catalyst which comprises from 5 to 95% by weight of CuO, preferably from 30 to 70% by weight of CuO, and from 5 to 95% by weight of Al₂O₃, preferably from 30 to 70% by weight of Al₂O₃, and from 0 to 60% by weight, preferably from 5 to 40% by weight, of ZnO.

The present invention relates to a process for preparing pyrrolidones. The synthesis is carried out by catalytic hydrogenation in the gas phase of substrates selected from the group consisting of derivatives of maleic acid and succinic acid and these acids themselves. The nitrogen building block present in the pyrrolidone can already be present in these substrates, otherwise ammonia or primary amine may be added in this synthesis should this nitrogen building block not be present. The process of the present invention makes it possible to prepare pyrrolidones which, if desired, are N-alkylated, but whether or not they are, the pyrrolidones can also bear one or more alkyl substituents on the carbon atoms of the ring.

The preparation of pyrrolidones by hydrogenation of maleic anhydride (MA) or succinic anhydride (SA) or the corresponding open-chain acids or esters in the presence of ammonia or primary amines is known per se.

Thus, EP-A 745 589 describes a process for preparing pyrrolidones, which may be N-substituted, in which MA, ammonia or a primary amine and hydrogen are reacted with one another over a supported catalyst. The catalyst comprises both rhenium and palladium in metallic or bound form.

It has been able to be shown that firstly BA and subsequently γ-butyrolactone (GBL) are formed in the hydrogenation of MA to tetrahydrofuran. These products can be converted by further hydrogenation into 1,4-butanediol (BDO) and subsequently into tetrahydrofuran (THF).

If the abovementioned starting materials are reacted with ammonia or primary amines under hydrogenating conditions, many competing reactions therefore occur, as a result of which the selectivity to the desired pyrrolidones is generally very low.

In addition, the catalysts used frequently contain chromium, generally in the form of chromium oxide. However, owing to the toxicity of chromium, it is desirable to develop catalysts which are free of chromium and give good yields and selectivities in respect of the desired pyrrolidone.

A further disadvantage of the processes used hitherto for preparing pyrrolidones or the catalysts employed for this purpose is that prepurified MA or a derivative thereof generally has to be used as starting material. The starting material therefore has to be freed of impurities in frequently complicated processes after it has been prepared. MA is prepared by partial oxidation of particular hydrocarbons, namely benzene, butene mixtures or n-butane, with preference being given to using the latter. The crude product of the oxidation comprises the desired MA together with, in particular, by-products such as water, carbon monoxide, carbon dioxide, unreacted starting hydrocarbons and also acetic and acrylic acids. These by-products are formed when using any of the abovementioned hydrocarbons. The by-products are usually separated off by means of complicated processes, for example by distillation. This purification has been found to be necessary because, in particular, the catalysts used in the preparation of pyrrolidone from MA with addition of ammonia or amine under hydrogenating conditions are generally sensitive to such impurities. The deactivation of the catalysts is a problem even when using purified MA, since deposition of polymerization products of MA on the catalyst generally makes it necessary for the catalyst to be regenerated at relatively short intervals, frequently about 100 hours. The tendency for deactivation is increased further in the presence of polymerizable compounds, for example acrylic acid.

EP-A 545 150 discloses a process for preparing N-organo-substituted pyrrolidones, in particular N-methylpyrrolidone, from appropriate dicarboxylic acid derivatives. The catalyst used in this process comprises at least one element of the first, seventh or eighth transition group of the Periodic Table of the Elements. As nitrogen building block, it is possible to use a primary amine having the desired organic substituent. However, it is also possible to use a mixture of a corresponding secondary and/or tertiary amine with the primary amine. The reaction is carried out with addition of water and/or ammonia. In an example, MA is reacted with methylamine and hydrogen with addition of water at 200 bar to form N-methylpyrrolidone, with a catalyst comprising 50% by weight of CuO and 50% by weight of Al₂O₃ being used. The yield is only 38%.

JP 63-27476 discloses a process for preparing pyrrolidones by gas-phase hydrogenation of imides of maleic acid or succinic acid, preferably succinic acid. Catalysts used are based on Cu and may further comprise an oxide of Cr, Mg or Zn. Pyrrolidone yields of not more than 66% are achieved in the process (according to the examples) and a mixture of succinimide in butyrolactone in a ratio of 20:80 is always used.

It is an object of the present invention to provide a process which makes it possible to prepare unsubstituted or substituted pyrrolidones in high yields and with high selectivities by reaction of maleic anhydride or a related compound. The catalysts used should be free of chromium and make it possible to use an MA or a related compound which does not have to be subjected to complicated prepurification as starting material. An MA or a related compound in a quality as is obtained immediately after its preparation should preferably be able to be used.

We have found that this object is achieved by a process for preparing pyrrolidone, which may be N-substituted, by hydrogenation in the gas phase under anhydrous conditions of a substrate selected from among C₄-dicarboxylic acids and their derivatives, with or without addition of ammonia or primary amines and using a Cr-free catalyst which comprises from 5 to 95% by weight of CuO, preferably from 30 to 70% by weight of CuO, and from 5 to 95% by weight of Al₂O₃, preferably from 30 to 70% by weight of Al₂O₃, and from 0 to 60% by weight, preferably from 5 to 40% by weight, of ZnO.

For the purposes of the present invention, the term “C₄-dicarboxylic acids and their derivatives” refers to maleic acid and succinic acid which may be unsubstituted or bear one or more C₁-C₆-alkyl substituents, and also the monoesters and diesters, anhydrides and imides of these unsubstituted or alkyl-substituted acids. Examples-include monomethyl maleate, dimethyl maleate, maleic anhydride, succinic anhydride, citraconic anhydride, succinimide, N-methylsuccinimide, N-butylsuccinimide, maleimide and N-methylmaleimide. If imides are used as starting materials, no ammonia or primary amine is added. These imides can have been prepared in a preceding step by reaction of ammonia or the desired primary amine with the C₄-dicarboxylic acid or its derivative without addition of hydrogen.

Preference is given to using the anhydrides, in particular maleic anhydride or succinic anhydride, in the reaction according to the present invention. The most preferred substrate is maleic anhydride (MA).

In a preferred embodiment, the process of the present invention is carried out by firstly hydrogenating the C₄-dicarboxylic acid and/or derivative thereof, preferably maleic anhydride, by means of hydrogen in the presence of the catalysts employed according to the present invention. Ammonia or the respective amines are only introduced when the carbon-carbon double bond of the respective starting material has been predominantly or fully hydrogenated and the hydrogenation mixture accordingly consists predominantly or entirely of succinic acid, succinic esters and/or succinic anhydride. This process is preferably carried out in a single reactor, with ammonia or the primary amine only being introduced into the reactor at a place or at a point in time at which the above-described hydrogenation of the starting material has already occurred. However, the process of the present invention can also be carried out in two reactors, with the hydrogenation of the starting material occurring in the first reactor and the reaction with ammonia or the primary amine occurring in the second reactor.

The nitrogen building block which is reacted is in the simplest case ammonia. If an N-substituted pyrrolidone is to be prepared, the nitrogen building block used is a primary amine which bears a substituent selected from the group consisting of substituted and unsubstituted cyclic and acyclic, branched and unbranched aliphatic hydrocarbon radicals having from 1 to 20 carbon atoms, preferably from 1 to 12 carbon atoms. The substituents which may be present on the aliphatic hydrocarbon are preferably selected from the group consisting of aromatic radicals, in particular the phenyl radical, and hydroxyl groups, halogens and alkoxy radicals. Examples of preferred primary amines are methylamine, ethylamine, n-propylamine, n-butylamine, n-decylamine, n-dodecylamine, cyclohexylamine, benzylamine and ethanolamine. If an imide is used as starting material, this may be N-substituted; the substituents may be as indicated above.

In place of pure primary amines, it is also possible to use mixtures of primary amines with the corresponding secondary and tertiary amines, e.g. methylamine, dimethylamine and trimethylamine. This embodiment is not preferred. A sufficient concentration of the primary amine is maintained by measures known to those skilled in the art.

The hydrogenation catalysts used according to the present invention comprise, prior to activation with hydrogen, copper oxide and aluminum oxide or copper oxide, aluminum oxide and zinc oxide. The copper oxide content is from 5 to 95% by weight and the aluminum oxide content is from 95 to 5% by weight. Preference is given to from 30 to 70% by weight of copper oxide and from 70 to 30% by weight of aluminum oxide, particularly preferably from 50 to 60% by weight of copper oxide and from 50 to 40% by weight of aluminum oxide. Such catalysts may further comprise up to 60% by weight of zinc oxide, preferably from 5 to 40% by weight of zinc oxide, in addition to copper oxide and aluminum oxide in the stated amounts.

The catalysts used according to the present invention, which are Cr-free, can optionally further comprise one or more additional metals or compounds thereof, preferably an oxide, from groups 1 to 14 (IA to VIIIA and IB to IVB of the old IUPAC nomenclature) of the Period Table of the Elements. If such a further oxide is employed, preference is given to using TiO₂, ZrO₂, SiO₂ and/or MgO.

It has been found that the catalysts used according to the present invention give high selectivities to the desired pyrrolidones and result in high yields. Unlike the process described in EP-A 545 150, the present process is carried out under anhydrous conditions.

This means that at most only the water formed by hydrogenation of a carbonyl group and any partial overhydrogenation of the second carbonyl group is present and water is not added deliberately. Merely the water impurities present in the starting materials may be present.

In addition, the catalysts used can further comprise an auxiliary in an amount of from 0 to 10% by weight. For the purposes of the present invention, auxiliaries are organic and inorganic substances which contribute to improved processing during catalyst production and/or to an increase in the mechanical strength of the shaped catalyst bodies. Such auxiliaries are known to those skilled in the art; examples include graphite, stearic acid, silica gel and copper powder.

The catalysts can be produced by methods known to those skilled in the art. Preference is given to processes in which the copper oxide is obtained in finely divided form intimately mixed with the other constituents, particularly preferably precipitation reactions. Here, precursor compounds dissolved in a solvent are precipitated in the presence of further soluble metal compounds or metal compounds suspended in the solvent by means of a precipitant, filtered off, washed, dried and, if desired, calcined.

The starting materials can be processed by known methods to give the shaped bodies, for example extrusion, tableting or by agglomeration methods, with or without addition of auxiliaries.

As an alternative, catalysts suitable for use according to the invention can also be produced, for example, by application of the active component to a support, for example by impregnation or vapor deposition. Furthermore, catalysts used according to the invention can be obtained by shaping a heterogeneous mixture of active component or a precursor compound thereof with a support component or a precursor compound thereof.

In the hydrogenation according to the present invention, in which not only MA but also other C₄-dicarboxylic acids defined above or derivatives thereof can be used as starting material, the catalyst is employed in reduced, activated form. Activation is achieved by means of reducing gases, preferably hydrogen or hydrogen/inert gas mixtures, either before or after installation in the reactor in which the process of the present invention is carried out. If the catalyst has been installed in oxidic form in the reactor, the activation can be carried out either before the hydrogenation according to the present invention is commenced in the plant or during start-up, i.e. in situ. Separate activation prior to start-up of the plant is generally carried out by means of reducing gases, for example hydrogen or hydrogen/inert gas mixtures, at elevated temperatures, preferably from 100 to 300° C. In in-situ activation, activation is carried out during running-up of the plant by contact with hydrogen at elevated temperature.

The catalysts are used as shaped bodies. Examples include extruded rods, extruded ridged rods, other extruded shapes, pellets, rings, spheres and granules.

The BET surface area of the copper catalysts in the oxidic state is from 10 to 400 m²/g, preferably from 15 to 200 m²/g, in particular from 20 to 150 m²/g. The copper surface area (measured by N₂O decomposition) of the reduced catalyst in the installed state is >0.2 m²/g, preferably >1 m²/g, in particular >2 m²/g.

In one variant of the invention, catalysts having a defined porosity are used. As shaped bodies, these catalysts have a pore volume of ≧0.01 ml/g for pore diameters of >50 nm, preferably ≧0.025 ml/g for pore diameters of >100 nm and in particular ≧0.05 ml/g for pore diameters of >200 nm. Furthermore, the ratio of macropores having a diameter of >50 nm to the total pore volume for pores having a diameter of >4 nm is >10%, preferably >20%, in particular >30%. High pyrrolidone yields and selectivities can often be achieved by use of these catalysts. The porosities mentioned were determined by mercury intrusion in accordance with DIN 66133. The data were evaluated in the pore diameter region from 4 nm to 300 μm.

The catalysts used according to the present invention generally have a satisfactory operation life. If the activity and/or selectivity of the catalyst should nevertheless drop during operation, it can be regenerated by methods known to those skilled in the alt. These include, preferably, reductive treatment of the catalyst in a stream of hydrogen at elevated temperature. If desired, the reductive treatment may be preceded by an oxidative treatment. In this case, a gas mixture comprising molecular oxygen, for example air, is passed through the catalyst bed at elevated temperature. It is also possible to wash the catalyst with a suitable solvent, for example ethanol, THF or GBL, and subsequently to dry it in a gas stream.

Furthermore, adherence to certain reaction parameters is necessary in order to achieve the pyrrolidone selectivities according to the present invention,

An important parameter is adherence to a suitable reaction temperature. One way of achieving this is by means of a sufficiently high inlet temperature of the starting materials. This is from 200 to 300° C. preferably from 210 to 280° C.

The space velocity over the catalyst in the hydrogenation according to the present invention is in the range from 0.01 to 1.0 kg of starting material/l of catalyst ° hour. In the case of a possible but not preferred recirculation of intermediate formed by incomplete hydrogenation when MA is used as starting material, for example succinimide or an N-substituted succinimide, the space velocity over the catalyst is the sum of fresh starting material fed in and recirculated intermediate. If the space velocity over the catalyst is increased beyond the specified region, an increase in the proportion of intermediate in the hydrogenation product is generally observed. The space velocity over the catalyst is preferably in the range from 0.02 to 1, in particular from 0.05 to 0.5, kg of starting material/l of catalyst • hour. Here, the term “starting material” refers to the starting materials which are fed into the process of the present invention and have functions which are hydrogenated during the course of the process, e.g. C═O or C═C double bonds. Amines or ammonia used are generally not encompassed by the term “starting material” in connection with the space velocity over the catalyst. In the case of recirculation, the term starting material also encompasses hydrogenation product which is initially formed and is then hydrogenated further after recirculation to form product, i.e., for example, N-methylsuccinimide in the case of the use of MA and methylamine in the hydrogenation reaction.

The hydrogen/starting material molar ratio is likewise a parameter which has an important influence on the product distribution and on the economics of the process of the present invention. From an economic point of view, a low hydrogen/starting material ratio is desirable. The lower limit is 3, but higher hydrogen/starting material molar ratios of from 20 to 400 are generally employed. The use of the above-described catalysts to be used according to the present invention and adherence to the above-described temperature values allows the use of favorable, low hydrogen/starting material ratios, preferably from 20 to 200, more preferably from 40 to 150. The most favorable range is from 50 to 100.

To set the hydrogen/starting material molar ratios used according to the present invention, part, advantageously the major part, of the hydrogen is circulated. For this purpose, the circulating gas compressors known to those skilled in the art are generally used,

The amount of hydrogen consumed chemically by the hydrogenation is replaced. In a preferred embodiment, part of the circulating gas is bled off in order to remove inert compounds, for example n-butane. The circulated hydrogen can also, if appropriate after preheating, be utilized for vaporizing the starting material stream,

The molar ratio of the starting materials C₄-dicarboxylic acid and/or derivative thereof to ammonia or primary amine is 1:5, preferably 1:3, particularly preferably 1:15.

It may be advantageous to use a solvent in the gas-phase hydrogenation. Possible solvents are, for example, ethers such as dioxane, tetrahydrofuran, alcohols such as methanol or hydrocarbons such as cyclohexane.

The volume flow of the reaction gases, generally expressed as GHSV (gas hourly space velocity), is also an important parameter in the process of the present invention. The GHSV in the process of the present invention is from 100 to 10 000 standard m³/m³h, preferably from 1 000 to 3 000 standard m³/m³h, in particular from 1 100 to 2 500 standard m³/m³h.

The pressure at which the hydrogenation according to the present invention is carried out is from 1 to 100 bar, preferably from 1 to 50 bar, in particular from 1 to 20 bar.

All products which do not condense or do not condense completely on cooling the gas stream leaving the hydrogenation reactor are circulated together with the circulating hydrogen gas. These are, in particular, water, ammonia and amines and by-products such as methane and butane. The cooling temperature is from 0 to 60° C., preferably from 20 to 45° C.

Suitable types of reactor are all apparatuses suitable for heterogeneously catalyzed reactions involving gaseous starting material and product streams. Preference is given to tube reactors, shaft reactors or reactors with internal removal of heat, for example shell-and-tube reactors; the use of a fluidized bed is also possible. Particular preference is given to using shell-and-tube reactors. A plurality of reactors can be connected in parallel or in series. In principle, additional starting material can be fed in between the catalyst beds. Intermittent cooling between or in the catalyst beds is also possible. When fixed-bed reactors are used, dilution of the catalyst by inert material is possible.

The gas stream leaving the reactor is cooled to from 10 to 60° C. The reaction products are condensed on cooling and are passed to a separator. The uncondensed gas stream is taken off from the separator and is passed to the circulating gas compressor. A small amount of circulating gas is bled off. The condensed reaction products are continuously taken from the system and passed to work-up, which can be carried out, for example, by distillation.

In the process of the present invention, starting materials to be hydrogenated having differing purities can be used in the hydrogenation reaction. It is of course possible to use a starting material of high purity, in particular MA, in the hydrogenation reaction. However, the catalyst used according to the present invention and the other reaction conditions chosen according to the present invention also make it possible to use starting materials of low purity, in particular MA which is contaminated by the usual compounds formed in the oxidation of benzene, butenes or n-butane and also by any further components. Thus, in a further embodiment, the process of the present invention can include a preceding step which comprises the preparation of the C₄-dicarboxylic acid and/or derivative thereof by partial oxidation of a suitable hydrocarbon and the separation of this starting material from the product stream obtained thereby.

In particular, the C₄-dicarboxylic acid is MA Preference is given to using MA which originates from the partial oxidation of hydrocarbons. Suitable hydrocarbon streams are benzene, C₄-olefins (e.g. n-butenes, C₄ raffinate streams) or n-butane. Particular preference is given to using n-butane, since it is an inexpensive, economical starting material. Processes for the partial oxidation of n-butane are described, for example, in Ullmann's Encyclopedia of Industrial Chemistry, 6th Edition, Electronic Release, Maleic and Fumaric Acids-Maleic Anhydride.

The reaction mixture obtained in this way is then taken up in a suitable organic solvent or solvent mixture which has a boiling point at atmospheric pressure which is at least 30° C. higher then that of MA.

This solvent (absorption medium) is brought to a temperature in the range from 20 to 160° C., preferably from 30 to 80° C. The gas stream comprising maleic anhydride from the partial oxidation can be brought into contact with the solvent in a variety of ways: (i) passing the gas stream into the solvent (e.g. via gas inlet nozzles or sparging rings), (ii) spraying the solvent into the gas stream and (iii) countercurrent contact between the upward flowing gas stream and the downward flowing solvent in a column provided with trays or packing. In all three variants, the apparatuses known to those skilled in the art for gas absorption can be used. When choosing the solvent to be used, it should be ensured that it does not react with the starting material, for example the preferably used MA. Suitable solvents are: tricresyl phosphate, dibutyl maleate, high molecular weight waxes, aromatic hydrocarbons having a molecular weight of from 150 to 400 and a boiling point above 140° C., for example dibenzylbenzene; dialkyl phthalates having C₁-C₈-alkyl groups, for example dimethyl phthalate, diethyl phthalate, dibutyl phthalate, di-n-propyl phthalate and diisopropyl phthalate; di-C₁-C₄-alkyl esters of other aromatic and aliphatic dicarboxylic acids, for example dimethyl 2,3-naphthalenedicarboxylate, dimethyl 1,4-cyclohexanedicarboxylate, methyl esters of long-chain fatty acids having, for example, from 14 to 30 carbon atoms, high-boiling ethers, for example the dimethyl ether of polyethylene glycol, for example tetraethylene glycol dimethyl ether.

The use of phthalates is preferred.

The solution resulting from the treatment with the absorption medium generally has an MA content of from about 5 to 400 gram per liter,

In a preferred embodiment of the present invention, it is also possible, as described in WO 01/27.058 A1, to carry out a partial condensation on the reaction product, use the resulting liquid MA without further purification for the hydrogenation according to the present invention and take up the remaining MA present in the gas phase in an organic solvent, as described above, and, if desired, use this MA in the hydrogenation after removal of the solvent. The process of partial condensation/extraction of MA and feeding the MA into the subsequent reaction stage as described in WO 01/27.058 is an integral part of the present invention and is hereby incorporated by reference.

The tailgas stream remaining after the treatment with the absorption medium comprises mainly the by-products of the preceding partial oxidation, e.g. water, carbon monoxide, carbon dioxide, unreacted butanes, acetic acid and acrylic acid. The tailgas stream is virtually free of MA.

The dissolved MA is subsequently stripped from the absorption medium. This is carried out using hydrogen at the pressure of the subsequent hydrogenation or not more than 10% above it or alternatively under reduced pressure with subsequent condensation of remaining MA. The stripping column is operated at a temperature profile resulting from the boiling points of MA at the top and virtually MA-free absorption medium at the bottom of the column under the respective column pressure and the dilution with carrier gas employed (in the first case, using hydrogen). In the case of direct stripping with hydrogen, a temperature at the top of 130° C. and a pressure of 5 bar are employed.

To prevent losses of solvent, rectification internals may also be present above the feed point for the crude MA stream. The virtually MA-free absorption medium taken off from the bottom is fed back into the absorption zone. In the case of direct stripping with hydrogen, a virtually saturated gas stream comprising MA in hydrogen is taken off at the top of the column at 180° C. and a pressure of 5 bar. The H₂/MA ratio is from about 20 to 400. Otherwise, the condensed MA is pumped into a vaporizer and there vaporized into the circulating gas stream.

The MA/hydrogen stream further comprises by-products formed in the partial oxidation of n-butane, butenes or benzene by means of oxygen-containing gases and also absorption medium which has not been separated off. These components are, in particular, acetic acid and acrylic acid as by-products, water, maleic acid and the dialkyl phthalates which are preferably used as absorption medium. The MA contains acetic acid in amounts of from 0.01 to 1% by weight, preferably from 0.1 to 0.8% by weight, and acrylic acid in amounts of from 0.01 to 1% by weight, preferably from 0.1 to 0.8% by weight, based on MA. In the hydrogenation step, acetic acid and acrylic acid are wholly or partly hydrogenated to ethanol or propanol. The maleic acid content is from 0.01 to 1% by weight, in particular from 0.05 to 0.3% by weight, based on MA.

If dialkyl phthalates are used as absorption media, their concentration in the MA depends strongly on correct operation of the stripping column, in particular the enrichment section. Phthalate contents of up to 1.0% by weight, in particular up to 0.5% by weight, should not be exceeded under appropriate operation, since otherwise the consumption of absorption medium becomes too high.

The hydrogen/maleic anhydride stream obtained in this way is then admixed with ammonia or a primary amine and hydrogenated in the process of the present invention to give the desired, if desired N-substituted, pyrrolidone.

The invention is illustrated by the following examples.

### EXAMPLE 1

### Preparation of N-Methylpyrrolidone

a) Catalyst Activation

The catalyst used in examples 1 to 3 consists of 50% by weight of CuO and 50% by weight of Al₂O₃ before activation. Before commencement of the reaction, it is subjected to a treatment with hydrogen at 180° C. The catalyst is activated successively using the mixtures of hydrogen and nitrogen indicated in table 1 for the indicated times at atmospheric pressure.

TABLE 1

| | Time | Hydrogen | Nitrogen |
|---|---|---|---|
| Composition | (minutes) | (standard l/h) | (standard l/h) |
| 50% by weight of CuO, | 120 | 10 | 550 |
| 50% by weight of Al₂O₃ | 30 | 25 | 400 |
| | 15 | 60 | 100 |
| | 180 | 60 | 0 |

b) Hydrogenation Apparatus and Hydrogenation Procedure:

The continuously operated hydrogenation is carried out in a vertical, electrically heated tube reactor made of quartz, in which the activated catalyst is present in the form of 3×3 mm pellets. Maleic anhydride is fed in from the top as a melt, and methylamine together with hydrogen in gaseous form are likewise introduced from the top. The products leaving the lower end of the reactor after passage over the catalyst are cooled. The liquid crude product is analyzed by gas chromatography. The starting materials, reaction conditions, yields, conversions and selectivities are summarized in table 2. The molar ratio of maleic anhydride to hydrogen is 1:10.

TABLE 2

| | | | Space | | | | | | |
|---|---|---|---|---|---|---|---|---|---|
| | | | velocity | | | | | | |
| | | | over the | Molar | | | | | |
| | | Catalyst | catalyst | ratio of | | | NMP | | |
| | Starting | [% by | [kg/l of | MA/ | Temperature | Pressure | yield¹⁾ | Conversion | NMP |
| Example | compound | weight]²⁾ | cat · h] | CH₃NH₂ | [° C.] | [bar] | [%] | [%] | selectivity |
| 1 | Maleic | 50% by | 0.1 | 1:1.5 | 250 | 1 | 70 | 100 | 70 |
| | anhydride | weight | | | | | | | |
| | | of CuO, | | | | | | | |
| | | 50% by | | | | | | | |
| | | weight | | | | | | | |
| | | of | | | | | | | |
| | | Al₂O₃ | | | | | | | |
| 2 | Succinic | see | 0.1 | 1:1.5 | 245 | 1 | 65 | 70 | 93 |
| | anhydride | above | | | | | | | |
| 3 | N- | see | 0.1 | no | 245 | 1 | 90 | 95 | 95 |
| | methyl- | above | | CH₃NH₂ | | | | | |
| | succinimide | | | | | | | | |

¹⁾N-methylpyrrolidone
²⁾prior to activation with hydrogen

We claim:

1. A process for preparing pyrrolidone, which may be N-substituted, by hydrogenation in the gas phase under anhydrous conditions of a substrate selected from among C₄-dicarboxylic acids and their derivatives, with or without addition of ammonia or primary amines and using a Cr-free catalyst which comprises from 5 to 95% by weight of CuO, preferably from 30 to 70% by weight of CuO, and from 5 to 95% by weight of Al₂O₃, preferably from 30 to 70% by weight of Al₂O₃, and from 0 to 60% by weight, preferably from 5 to 40% by weight, of ZnO.

2. A process as claimed in claim 1, wherein the catalyst comprises one or more metals or compounds thereof, preferably an oxide, from groups 1 to 14 of the Periodic Table of the Elements, preferably a compound selected from the group consisting of TiO₂, ZrO₂, SiO₂ and MgO.

3. A process as claimed in claim 1 or 2, wherein the C₄-dicarboxylic acid or the derivative thereof is selected from the group consisting of maleic acid, maleic anhydride, succinic acid, succinic anhydride and substituted and unsubstituted maleimide and succinimide, in particular maleic anhydride.

4. A process as claimed in any of claims 1 to 3, wherein the primary amine or the imide bears a substituent selected from the group consisting of substituted and unsubstituted cyclic and acyclic, branched and unbranched aliphatic C₁-C₂₀-hydrocarbons, preferably C₁-C₁₂-hydrocarbon groups.

5. A process as claimed in any of claims 1 to 4, wherein the aliphatic groups may bear one or more substituents selected from the group consisting of phenyl groups, halogens, hydroxyl groups and alkoxy groups, and the aliphatic groups are preferably selected from among methyl, ethyl, n-propyl, n-butyl, n-decyl, n-dodecyl, cyclohexyl, benzyl and ethylol.

6. A process as claimed in any of claims 1 to 5, wherein an unsaturated dicarboxylic acid or a derivative thereof, preferably maleic acid, a maleic ester and/or maleic anhydride, is used as starting material and the ammonia or the primary amine is added to the reaction mixture only after partial or complete hydrogenation of the olefinic double bond of the starting material or materials.

7. A process as claimed in any of claims 1 to 6, wherein the reaction is carried out at from 200 to 300° C., preferably from 210 to 280° C.

8. A process as claimed in any of claims 1 to 7 carried out at pressures of from 1 to 100 bar, preferably from 1 to 50 bar.

9. A process as claimed in any of claims 1 to 8 in which a fixed-bed reactor, preferably a tube reactor, a shaft reactor, a fluidized-bed reactor or a reactor with internal removal of heat, in particular a shell-and-tube reactor, is used.

10. A process as claimed in any of claims 1 to 9, wherein maleic anhydride is obtained by oxidation of benzene, C₄-olefins or n-butane, extraction of the maleic anhydride by means of a solvent from the crude product mixture obtained by oxidation and subsequent stripping from this solvent by means of hydrogen is used.

11. A process as claimed in claim 10, wherein maleic anhydride is condensed from the crude product mixture obtained by oxidation and is used without purification in the hydrogenation and the uncondensed maleic anhydride is then extracted from the crude product mixture by means of a solvent and is optionally used for the hydrogenation.

12. A process as claimed in claim 1, wherein a mixture of primary amine with the corresponding secondary and amine is used.
